(19) United States
(12) Patent Application Publication
(10) Pub. No.: US 2024/0293032 A1
(43) Pub. Date: Sep. 5, 2024

(54) SOFT STERNAL PHOTOPLETHYSMOGRAPHIC PATCH

(71) Applicant: Georgia Tech Research Corporation, Atlanta, GA (US)

(72) Inventors: Woon-Hong Yeo, Atlanta, GA (US); Nathan Zavanelli, Atlanta, GA (US)

(21) Appl. No.: 18/594,967

(22) Filed: Mar. 4, 2024

Related U.S. Application Data
(60) Provisional application No. 63/488,129, filed on Mar. 2, 2023.

(51) Int. Cl. A61B 5/02; A61B 5/00; A61B 5/28; G16H 15/00

(52) U.S. Cl. CPC A61B 5/02007; A61B 5/28; A61B 5/4818; A61B 5/6823; A61B 5/6833; A61B 5/7207; A61B 5/7267; A61B 5/7282; A61B 5/742; G16H 15/00; A61B 2560/0412; A61B 2562/166

(57) ABSTRACT

An exemplary system and method are disclosed that employ a wireless, fully integrated, soft sternal patch to capture PPG signals from the sternum, an anatomical region that exhibits a robust vasoconstrictive response while also providing a convenient sternal location for the integration of electrocardiography and seismocardiography sensors in a single, all-in-one device capable of multifaceted human health monitoring. With healthy control subjects, the device is is configured to detect vasoconstriction induced endogenously through controlled breathing exercises and exogenously through changes in ambient temperature. Furthermore, in overnight trials with sleep apnea patients, the device shows a high agreement in vasoconstriction detection with a commercial system, demonstrating its potential use in continuous, long-term vasoconstriction monitoring.

### RELATED APPLICATION

This application claims priority to, and the benefit of, U.S. Provisional Patent Application No. 63/488,129, filed Mar. 2, 2023, which is incorporated by reference herein in its entirety.

### BACKGROUND

Conventional blood oxygen oximeters employ optical-based PPG measurements. The devices have a form factor comprising a clamp that compresses the finger and takes reliable measurements.

Vasoconstriction is a crucial physiological process that serves as the body's primary blood pressure regulation mechanism and a key marker of numerous harmful health conditions.

The ability to detect vasoconstriction in real-time can be beneficial for detecting blood pressure, identifying sympathetic arousals, characterizing patient well-being, detecting sickle cell anemia attacks early, and for identifying complications caused by hypertension medications. However, because of venous pooling, vasoconstriction manifests weakly in traditional photoplethysmography (PPG) measurement locations, like the finger, toe, and ear. Although this issue can be addressed by applying near diastolic pressure to the finger, this measurement technique causes disruptions during daily activities.

There is a benefit to the measurement of vasoconstriction via photoplethysmography (PPG) measurements and having photoplethysmography measurements at the chest.

### SUMMARY

An exemplary flexible sternal photoplethysmographic (PPG) patch sensor is disclosed that can be placed at the sternal region of a patient or user for photoplethysmographic measurements, the sternal region being an anatomical region that exhibits a robust vasoconstrictive response. Photoplethysmographic sensing based on optical signals requires contact with the skin without any air gap. Conventional photoplethysmography has form factor benefits via a clip that can be secured to the finger. Such devices can not operate on a flat surface such as the sternum.

The exemplary flexible sternal photoplethysmographic patch sensor includes a flexible printed circuit board that includes the PPG sensor. One or more elastomer layers couple to the printed circuit board and dispose over the PPG sensor to generate a compression force around the PPG sensor and urge the PPG sensor toward the tissue to make conformal contact with the skin without any air gap. The patch sensor has a flat form factor that is attached to the hard region of the sternum while providing the desired compressive force.

The sternal photoplethysmographic patch sensor can include additional sensors, e.g., sound, electrical, and temperature, to provide concurrent ECG, HR, and respiration measurements. The exemplary flexible sternal PPG patch sensor can be used for sleep monitoring, heart condition monitoring, as well as general health monitoring, among others. In being placed at the sternum, it allows for the acquisition of PPG signals without affecting the use of the hand or fingers.

The photoplethysmographic patch sensor can be applied to other body location have a flat bone region, e.g., forehead, skull, etc.

In some embodiments, the photoplethysmographic patch sensor is configured as a wireless, fully self-contained, integrated, soft sternal patch to capture PPG signals from the sternum for the integration of electrocardiography and seismocardiography sensors in a single, all-in-one device capable of multifaceted human health monitoring. With healthy control subjects, the device is configured to detect vasoconstriction induced endogenously through controlled breathing exercises and exogenously through changes in ambient temperature. Furthermore, in overnight trials with sleep apnea patients, the device shows a high agreement in vasoconstriction detection with a commercial system, demonstrating its potential use in continuous, long-term vasoconstriction monitoring.

In one embodiment, provided is a system comprising: a processor; and a memory having instructions stored thereon, wherein the instructions when executed by the processor causes the processor to: receive a photoplethysmogram (PPG) signal data set acquired by a soft patch comprising a vasoconstriction sensor placed on a chest of a subject; and determine if the subject is experiencing vasoconstriction using the the PPG signal data set. The soft patch can compress the vasoconstriction sensor into the chest of the subject.

In some embodiments, the instructions, when executed by the processor, can further cause the processor to output if the subject is experiencing vasoconstriction in a graphical user interface or a report to be used for an evaluation of cardiovascular health in the subject.

In some embodiments, the soft patch can further include an electrocardiography sensor and/or a seismocardiography sensor.

In some embodiments, the soft patch can be placed on the sternum of the subject.

In some embodiments, the soft patch can further include an elastomer disposed over the vasoconstriction sensor. The elastomer can dampen motion artifacts in the PPG signal data caused by the movement of the chest of the subject.

In some embodiments, the soft patch can include wireless communication circuitry.

In some embodiments, the instructions, when executed by the processor, can further cause the processor to determine via a trained machine learning model if the subject is experiencing sleep apnea.

In some embodiments, the instructions, when executed by the processor, can further cause the processor to output if the subject is experiencing sleep apnea in a graphical user interface or a report to be used for an evaluation of sleep apnea in the subject.

In some embodiments, the system can be configured as a smartwatch or smartphone.

In some embodiments, the system can be implemented in cloud infrastructure.

In another embodiment, provided is a method comprising: receiving a PPG signal dataset acquired by a soft patch comprising a vasoconstriction sensor placed on a chest of a subject; and determining if the subject is experiencing vasoconstriction using the the PPG signal data set. The soft patch can compress the vasoconstriction sensor into the chest of the subject.

In some embodiments, the method can further include outputting if the subject is experiencing vasoconstriction in a graphical user interface or a report to be used for an evaluation of cardiovascular health in the subject.

In some embodiments, the soft patch can further include an electrocardiography sensor and/or a seismocardiography sensor.

In some embodiments, the soft patch can be placed on a sternum of the subject.

In some embodiments, the soft patch can further include an elastomer disposed over the vasoconstriction sensor. The elastomer can dampen motion artifacts in the PPG signal data caused by movement of the chest of the subject.

In some embodiments, the soft patch can include wireless communication circuitry.

In some embodiments, the method can further include determining via a trained machine learning model if the subject is experiencing sleep apnea.

In some embodiments, the method can further include outputting if the subject is experiencing sleep apnea in a graphical user interface or a report to be used for an evaluation of sleep apnea in the subject.

In another embodiment, provided is a non-transitory computer-readable medium having instructions stored thereon, wherein the instructions, when executed by a processor, cause the processor to: receive a photoplethysmogram (PPG) signal data set acquired by a soft patch comprising a vasoconstriction sensor placed on a chest of a subject; and determine if the subject is experiencing vasoconstriction using the PPG signal data set. The soft patch can compress the vasoconstriction sensor into the chest of the subject.

In some embodiments, the instructions, when executed by the processor, can further cause the processor to output if the subject is experiencing vasoconstriction in a graphical user interface or a report to be used for an evaluation of cardiovascular health in the subject.

### BRIEF DESCRIPTION OF DRAWINGS

FIGS. 1A-1B show an exemplary soft sternal patch configured to capture PPG signals from the sternum to provide robust vasoconstrictive response estimation.

FIGS. 2A-2C show operations of the soft sternal patch at the sternum.

FIG. 3 shows an example system configured to estimate vasoconstrictive response based on the soft sternal patch measurement.

FIGS. 4A-4B show an example method of estimating vasoconstrictive response.

FIG. 5A shows an example method of fabrication of the soft sternal patch.

FIGS. 6A-6B show an example fabricated soft sternal patch device.

FIG. 7A shows an image of the flexible device folded on a glass slide. FIG. 7B shows a calculation of the device bending stiffness (in terms of displacement of the tip during bending). FIGS. 7C-7E depict an experimental setup for a bending test showing the device with 0° (FIG. 7C), 900 (FIG. 7D), and 1600 (FIG. 7E) bending, respectively. FIG. 7F shows PPG signals recorded throughout 100 rounds of cyclic bending. The signals are comprised of light from the ambient environment. FIGS. 7G-7H depict signal waveforms at the beginning (FIG. 7G) and end (FIG. 7H) of the trial, demonstrating no deterioration. FIGS. 7I-7K depict FEA compression results. FIG. 7I shows FEA results for the soft device, showing the elastomeric board encapsulating around the PPG unit and skin with an applied force of 1.1N.

FIG. 7J shows comparison FEA results for a 0.4 mm flex PCB showing an inability to conform to the skin. FIG. 7K shows a simulation of stress applied to the user's skin. FIGS. 7L-7M depict a device biocompatibility assessment. FIG. 7L shows an image of the skin with the device area circled before device placement. FIG. 7M shows an image of the skin after 3 days of continuous wear, showing only a small red irritation in the circled device area.

FIGS. 8A-8B depict PPG signals during rest and perturbations of the skin induced by stretching and pulling for both the rigid (FIG. 8A) and soft (FIG. 8B) devices, respectively. FIG. 8C shows an image of the experimental setup. FIG. 8D shows an illustration of the measurement sites tested (triangles) with major vessels (red line) and major angiosomes (green). FIGS. 8E-8F depict charts of PPG signal repeatability (FIG. 8E) and percent vasoconstriction (FIG. 8F) during breath hold tests as a function of location (5 subjects, 3 trials, error bars of 1 SD). FIG. 8G shows representative ensembled PPG signals with annotated fiducial marks. FIGS. 8H-8K depict autocorrelation for the detection of PPG repeatability and signal quality. FIG. 8H shows a plot of the PPG autocorrelation defined at the 1 and 0 beat lags. The ratio of the successive lag peaks to the fundamental peak is the autocorrelative periodicity. FIGS. 8I-8K depict time series PPG signals plotted for −1 (FIG. 8I), 0 (FIG. 8J), and 1 (FIG. 8K) beat lags. FIGS. 8L-8M depict a demonstration of the Hilbert analytical envelope in a representative vasoconstriction. FIG. 8L shows the filtered signal, and FIG. 8M shows the corresponding Hilbert envelope, which represents the amplitude of each PPG wave. FIG. 8N shows a flow diagram of signal processing for percent vasoconstriction detection.

FIGS. 9A-9E depict vasoconstrictions in controlled experiments. FIG. 9A shows an image of the soft device on a subject's chest. FIGS. 9B-9C show representative vasoconstrictions observed upon the termination of breath hold. FIG. 9D shows summary statistics of percent vasoconstriction during breath hold experiments across subjects (5 trials, error bars of 1 SD). FIG. 9E shows PPG signals from the soft device during changes in ambient temperature, demonstrating a strong ability to detect exogenously induced vasoconstrictions.

FIG. 10A shows vasoconstriction in overnight trials with patients with symptomatic apnea, particularly data showing an overnight recording of the IR and Red PPG signals compared to a simultaneous recording of WatchPAT's PAT signals with annotated apnea events. FIGS. 10B-10H depict a quantitative assessment of apnea-induced vasoconstrictions. FIG. 10B shows a confusion matrix relating the identification of small (0%-15%), medium (15%-30%), and large (30%-50%) vasoconstrictions in the comparison of soft device and WatchPAT. FIG. 10C shows a Bland-Altman Diagram for the data reported in FIG. 10B (n=36, error bars of 1 SD). FIG. 10D shows a correlation plot for the data in FIG. 10B (n=36). FIG. 10E shows percent vasoconstriction for eight overnight trials (n=36, box 25-75 percentiles, whiskers extreme values). FIG. 10F shows percent vasoconstriction aggregated to include all eight nights, showing a non-zero vasoconstriction (n=231, box 25-75 percentiles, whiskers extreme values). FIG. 10G shows a boxchart comparison of vasoconstriction magnitude across all events. with P-value <1e-6. (n varies from 13-41, box 25-75 percentiles, whiskers extreme values). FIG. 10H shows an image of the soft device on a sleeping subject.

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entirety and to the same extent as if each reference was individually incorporated by reference.

### Example System

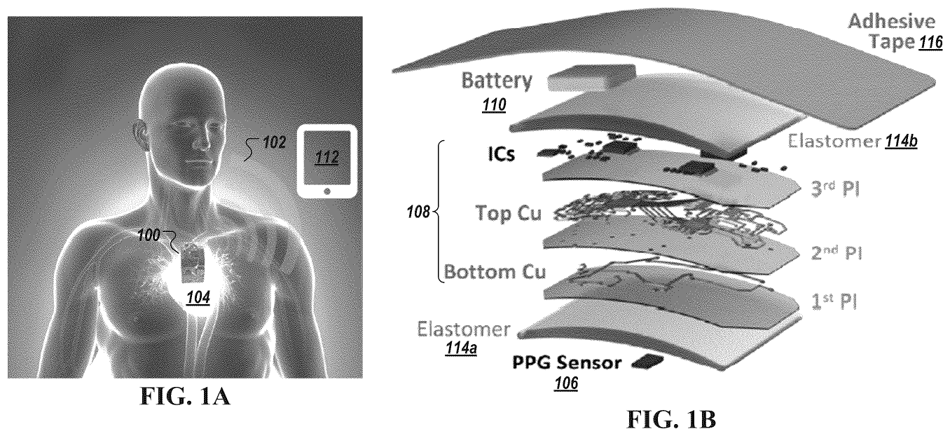

FIG. 1A shows an example soft patch 100 configured to capture PPG signals 102 from a subject to provide robust vasoconstrictive response estimation in accordance with an illustrative embodiment. In the example shown in FIG. 1A, the soft patch 100 is placed on the subject's sternum 104. It is considered that, in other embodiments, the soft sternal patch can alternatively be placed on any other suitable location on the subject's chest.

As shown in FIG. 1B, the soft patch 100 includes a PPG sensor 106 in direct contact with the subject's sternum 104. In some embodiments, there may be an additional material between the subject's sternum and the PPG sensor. It is considered that, in other embodiments, an alternate vasoconstriction sensor may be used. In some embodiments, the soft patch can further include an electrocardiography sensor and/or a seismocardiography sensor.

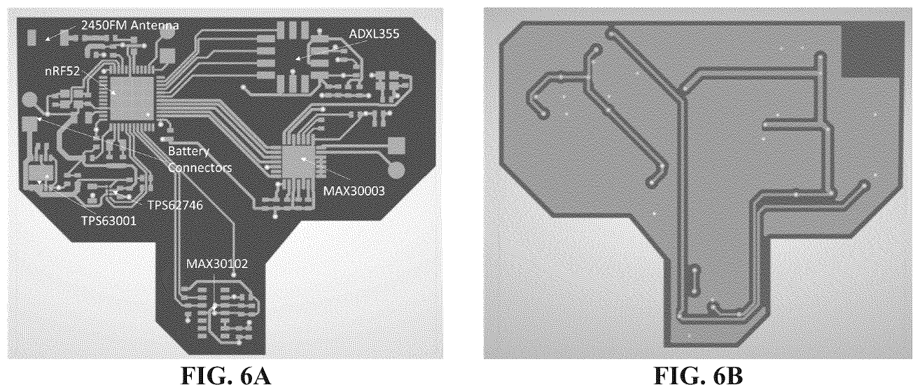

The PPG sensor 106 is in communication with the flexible device circuit 108 and powered by the battery 110. The flexible device circuit 108 is photolithographically patterned and ultrathin and functionalized with integrated components. An example flexible device circuit is shown in FIGS. 6A-6B. The flexible device circuit 108 includes wireless communication circuitry which allows communication of the PPG signals 102 to an external computing device. In some embodiments, the external computing device may be a smartwatch or a smartphone. In other embodiments, the external computing device may be cloud infrastructure.

The flexible device circuit 108 is embedded between two elastomer layers 114a, 114b. In some embodiments, each elastomer layer can include silicone, rubber, polyimide, and the like. In some embodiments, each elastomer layer can be made of Ecoflex 00-30. In some embodiments, each elastomer layer can be from about 1 mm to about 3 mm thick, e.g., as described herein. In some embodiments, the elastomer layer can be greater than 3 mm. The two elastomer layers 114a, 114b are configured to dampen motion artifacts in the PPG signal data, which can be caused by movement of the subject's sternum 104, for example, if the subject folds their arms across their chest and causes the skin surrounding the soft patch 100 to fold inward. In some embodiments, the soft patch may include only one elastomer layer.

The soft patch 100 includes a flexible printed circuit board 108 having the sensor 106 (shown as PPG sensor 106). The flexible printed circuit board 108 is embedded in the elastomer layer 114a, 114b having an asymmetric shape to place the flexible printed circuit board 108 in a bend configuration. To this end, once the soft patch 100 is secured to the subject's sternum 104 by an adhesive tape layer 116, the flexible printed circuit board 108 generates tension force to urge the PPG sensor 106 toward the skin/sternum.

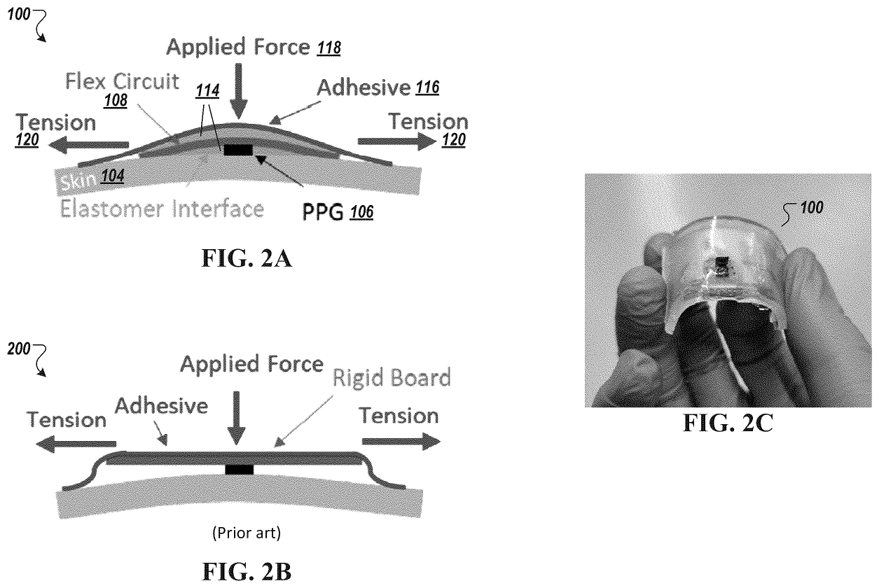

Indeed, the adhesive tape layer 116, in combination with the bend flexible printed circuit board and the asymmetrically shaped elastomer, are configured, collectively, to cause the soft path 100 to create an applied compression force 118 around the PPG sensor 106 as demonstrated in FIG. 2A. The lower elastomer layer (e.g., 114a) can create conformal contact with the subject's sternum 104 via natural van der Waals forces exhibited at the skin-elastomer interface, and the adhesive tape layer 116 is in contact with the subject's sternum 104 at the periphery of the soft patch 100. This applied compression force 118 allows highly conformal contact between the PPG sensor 106 and the subject's sternum 104, thereby providing sufficient skin-device contact to measure excellent quality PPG signals 102. Furthermore, the applied compression force 118 on the PPG sensor 106 can be modulated by the offset height of said PPG sensor relative to the two elastomer layers 114a, 114b and the tensile stress 120 in the adhesive tape layer 116. The adhesive tape layer 116 creates a consistent pressure on the PPG sensor 106 because it can rapidly increase stiffness at a specific tension. This allows the soft patch 100 to be placed and worn by a subject without needing a medical provider present to place said patch, making the soft patch 100 particularly accessible for at-home monitoring and care.

In contrast, FIG. 2B shows an example rigid patch 200 with a rigid board 202 used instead of the two elastomer layers 114a, 114b shown in FIG. 1B. The rigid patch 200 cannot provide the same skin-sensor contact and is highly susceptible to motion artifacts. This can drastically reduce the quality of the PPG signals recorded by the patch.

FIG. 2C shows a fabricated version of the soft patch 100.

### Example System

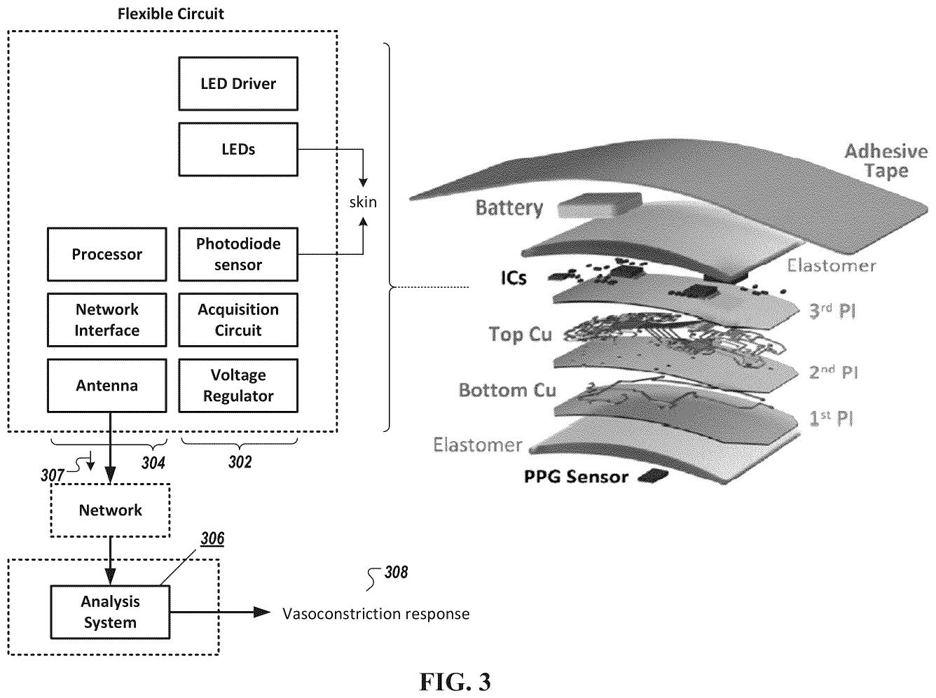

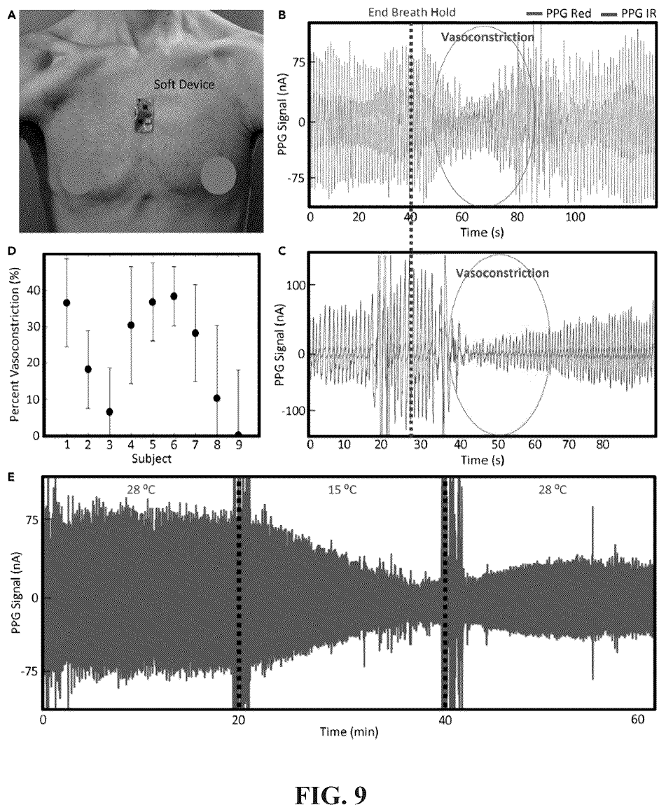

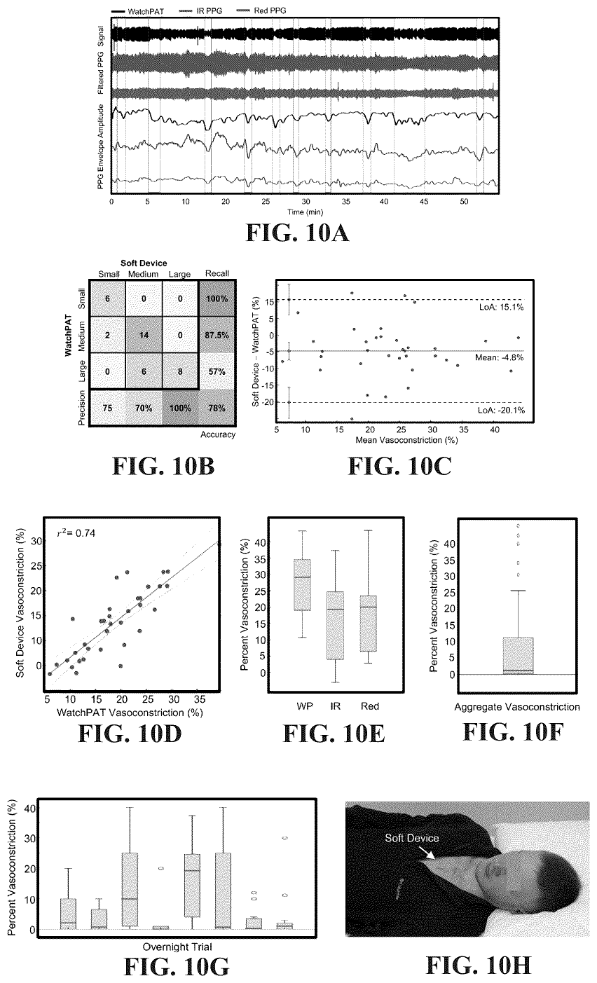

FIG. 3 shows an example example soft patch 100 (shown as 100′) configured to estimate vasoconstrictive response based on the soft sternal patch measurement. The example soft patch 100′ includes a flexible circuit board 108 comprising PPG sensor circuitries 302 and processing unit 304 to acquire the PPG signal 307 to provide to an analysis system 306 to provide a measure of vasoconstriction response 308. In the example shown in FIG. 3, the analysis system 306 is configured to analyze the acquired PPG signal 307, e.g., to determine vasoconstriction response. In other embodiments, the analysis system 306 is integrated into the example soft patch 100′. The analysis system 306 can provide the estimated vasoconstriction response to a graphical user interface or report, e.g., that can be used as a measure of sleep monitoring, heart condition monitoring, or general health monitoring, among others. Example of vasoconstriction response as provided herein, e.g., in relation to the description of FIGS. 9 and 10, among others.

### Example Method

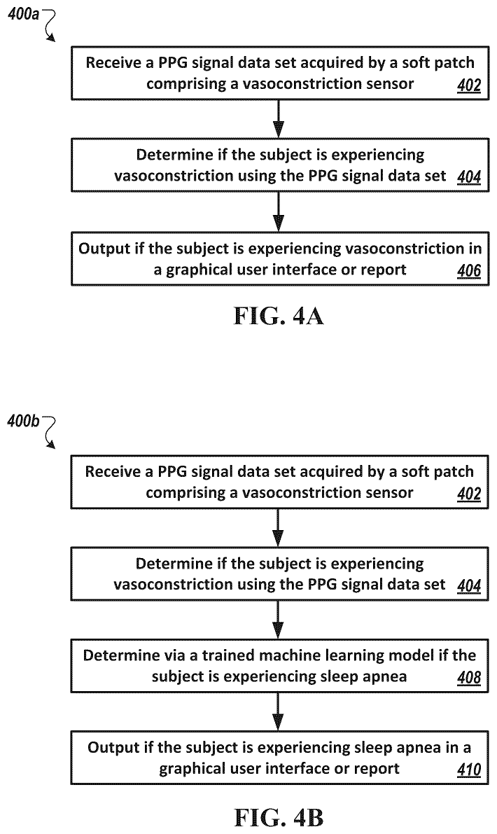

FIG. 4A shows an example method 400a of determining if a subject is experiencing vasoconstriction. First, a PPG signal data set acquired by a soft patch comprising a vasoconstriction sensor (e.g., the soft patch 100) is received 402. Using this data set, it is determined 404 if the subject is experiencing vasoconstriction. In some embodiments, this result may be output 406 in a graphical user interface or report, for example, to a smartphone or smartwatch. The method 400a can be used to detect isolated incidents of vasoconstriction or to continuously monitor vasoconstriction over a period of time (e.g., 1-3 days).

FIG. 4B shows an example method 400b of determining if a subject is experiencing sleep apnea. First, a PPG signal data set acquired by a soft patch comprising a vasoconstriction sensor (e.g., the soft patch 100) is received 402. Using this data set, it is determined 404 if the subject is experiencing vasoconstriction. Next, using a trained machine learning model, it is determined 408 if the subject is experiencing sleep apnea. In some embodiments, this result may be output 410 in a graphical user interface or report, for example, to a smartphone or srmartwarch. The method 400b can be used to detect isolated incidents of sleep apnea (or to continuously monitor sleep apnea over a period of time (e.g., 1-3 days).

### Example Fabrication and Design

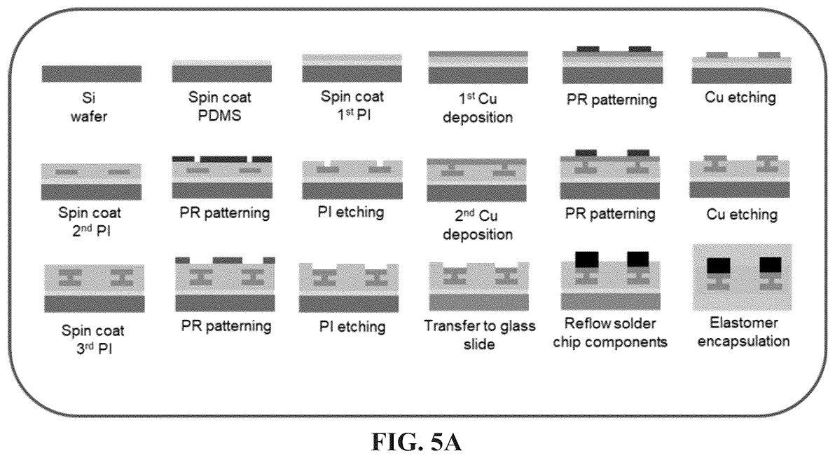

Device fabrication: The soft patch was microfabricated in a study using photolithographic patterning on a polydimethylsiloxane-coated Si wafer and two-stage transfer to an Ecoflex 00-30 elastomer, as described in FIG. 5A and demonstrated previously [27,28]. The circuit comprises top and bottom copper layers connected with etched vias and three polyimide dielectric layers. The integrated circuit components are soldered on top of pads exposed in the top copper layer.

Device reuse: The soft patch can be reused multiple times after undergoing a basic cleaning and an application of a new bottom adhesive layer. First, the tape is removed, and the device is sprayed with isopropyl alcohol. The device is then wiped clean with a wipe. Then it is wiped with ethyl alcohol. Finally, a thin layer of silbione is applied to the bottom of the device to form an adhesive layer with the skin.

Circuit information: The circuit (FIGS. 6A-6B) is equipped with a 150 mAh LiPo battery operating at 3.7V and downregulated to 3.3V by a TPS63001 voltage regulator and 1.8 V by a TPS62746 voltage regulator, respectively. The 3.3V is used solely to power the PPG sensor (MAX30102) LEDs. All other components operate on 1.8V. Battery life exceeds 10 h on a single charge with a power draw of <15 mA. The battery is recharged with a magnetic connector prior to each study. Central processing is handled by an nRF52832 BLE SoC, which communicates via I2C with the MAX30102 PPG sensor.

Experimental study for circuit bending: The circuit was progressively bent by a MARK-10 ESM303 motorized force measurement stand. The device was placed on two glass slides and bent down the interface between the slides. Device electrical perfor-mance was ensured by assessing Bluetooth transmission of PPG signals.

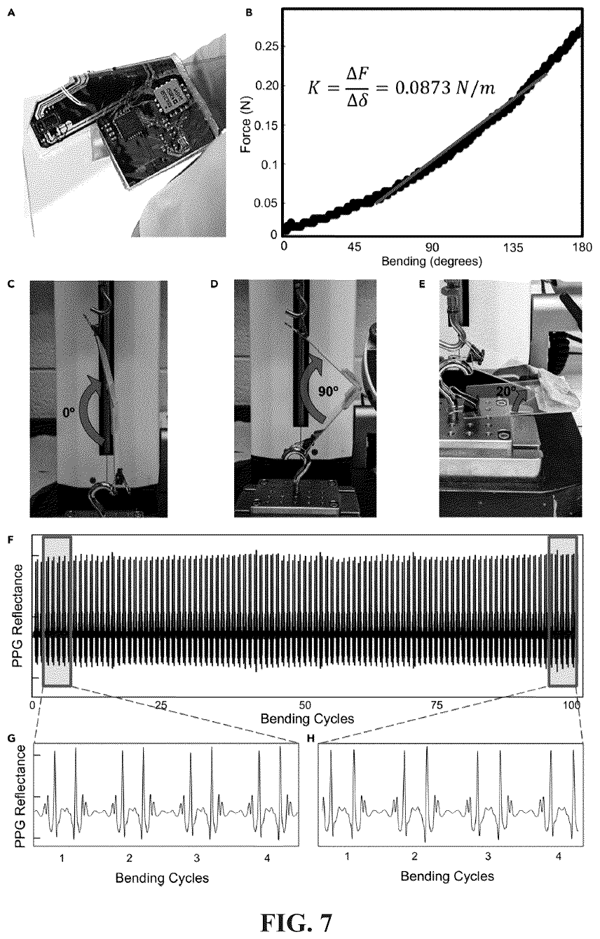

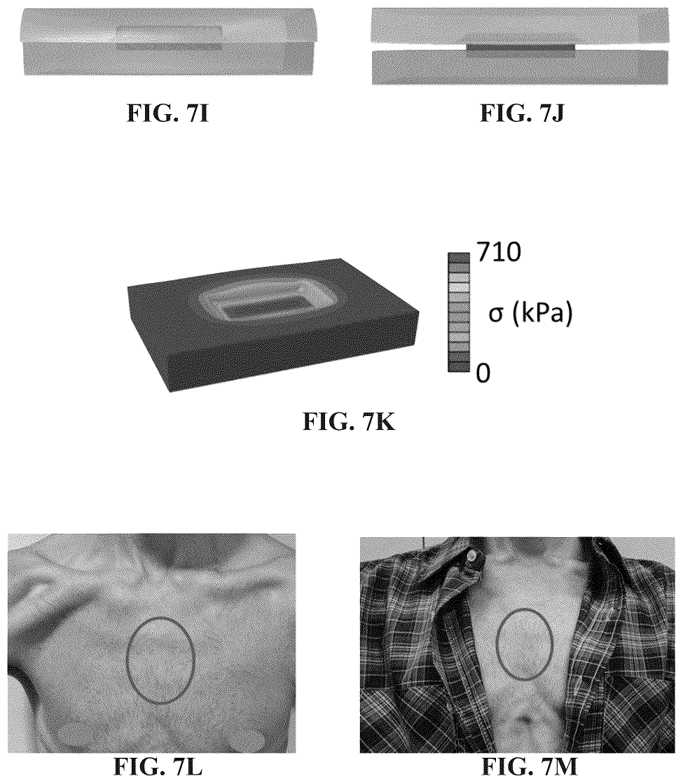

Finite element analysis for PPG pressure optimization: Finite element analysis (FEA) was conducted to simulate the conformal contact of the PPG unit to the skin for both a soft and rigid board (FIGS. 7I-7K). The skin consisted of a 100-mm-thick epidermis layer and a 2-mm-thick dermis layer. A PPG unit was partially embedded into a 2-mm-thick elastomer layer and poly-imide layer for the soft and rigid boards, respectively. The contact conditions were set between the PPG component and the simulated skin, and a linearly applied displacement was introduced on the top surface while the bottom was fixed. The elastomer and skin layers were meshed using C3D8R elements. The following material properties of Young's modulus (E) and Poisson's ratio (ν) were used for the model: Eₑₗₐₛₜₒₘₑᵣ=1 MPa and νₑₗₐₛₜₒₘₑᵣ=0.49; Eₚᵢ=4.0 GPa and vₚᵢ=0.34; E_{epidermis}=1 MPa and v_{epidermis}=0.48; E_{dermis}=0.2 MPa and v_{dermis}=0.48.

Sensing components: In the study, PPG is sensed through the MAX30102 sensor. Raw PPG waveforms are sampled at 200 Hz with a proprietary filter. This signal is oversampled by a factor of 4, resulting in a 50 Hz digital signal. Analog to digital con-version is handled by circuitry internal to the MAX30102, and the digital signal is 18 bits. In addition, the MAX30102 incorporates proprietary ambient light cancellation through track/hold circuitry.

Wireless communication: PPG data is broadcast from the nRF52832 BLE SoC in a 240-byte buffer following traditional GATT protocol. The Android tablet receives these BLE transmissions from the soft sternal patch device via a custom-de-signed GATT client app. Data are converted from binary to double, assigned a timestamp, and automat-ically saved in a CSV file in real time on the Android device. Data are plotted in real time so that the user can verify the function from the tablet. The data is saved in a CSV file, which may be exported from the android de-vice or sent via e-mail upon trial completion.

Signal processing and data assessment: All signal processing and data analysis was conducted in the MATLAB programming language. Real-time data display and processing on the tablet were implemented in Kotlin. Firmware was executed in embedded C. Signals were assessed in two ways. First, the periodicity was defined as the ratio of an auto-correlation peak for a subsequent beat to the primary peak. This periodicity is indicative of the repeatability of the signal and is a surrogate for signal-to-noise ratio. Second, the percentage vasoconstriction is calculated from the Hilbert analytic envelope, which tracks the amplitude of each PPG beat with time.

Calculation of autocorrelation for periodicity assessment: The autocorrelation for a given lag r(k) is a measurement of the correlation between the single variable time series waveform g(t) and g(t+k) where k=0, . . . , k is a random series. r(k) per Equation Set 1.

$$\begin{matrix}r ( k ) = \frac{{c}_{k}}{{c}_{0}} \\ where \\ {c}_{k} = \frac{1}{T} {\sum }_{t = 1}^{T} ( g ( t ) - {g ( t )}^{*} ) ( g ( t + k ) - {g ( t )}^{*} )\end{matrix} \qquad ( Eq . Set 1 )$$

In Equation Set 1, c₁ is the sample variance of the waveform and T is the length of the time interval examined. Here, the periodicity is defined as the ratio of the 4 largest autocorrelation peaks to the peak at time zero.

Calculation of hilbert analytic envelope: Let g(t) represent the bio-signal waveform, [symbol] (t) the Hilbert transform, and [symbol] (t) the analytic signal representation. [symbol] (t) is composed of g(t) and [symbol] (t) per Equation 2.

$$( t ) = g ( t ) + i ( t ) \qquad ( Eq . 2 )$$

[symbol] (t) can be defined using the Cauchy principal value (p.v.) by the convolution:

$$( t ) = \frac{1}{π} {\int }_{- ∞}^{∞} \frac{g ( τ )}{t - τ} d τ \qquad ( Eq . 3 )$$

In Equation 3, τ is introduced as a temporary variable for the purposes of integration. The analytic envelope function is defined as the absolute value of the analytic function per Equation 4.

$$\vert ( t ) \vert = \sqrt{( t ) {( t )}^{*}} \qquad ( Eq . 4 )$$

In Equation 4, [symbol] (t)* represents to complex conjugate of the analytic function.

In order to obtain the envelope in MATLAB, an ideal brick-wall sliding filter with a Kaiser window of length 500 (for a 50 Hz signal) and b of 8 is implemented. This filter ω(n) is defined per Equation 5.

$$𝔴 ( n ) = \frac{{I}_{o} ( β \sqrt{1 - {( \frac{2 n}{N - 1} - 1 )}^{2}} )}{{I}_{o} ( β )} \qquad ( Eq . 5 )$$

In Equation 5, N is the length of the filter, n is incremented from 0 to N−1, and Iₒ(⋅) is the zero-th order modified Bessel function of the first kind per Equation 6.

$${I}_{o} ( z ) = \frac{1}{2 π i} ∮ {e}^{( \frac{z}{2} ) ( \frac{t + 1}{t} )} {t}^{- 1} d t \qquad ( Eq . 6 )$$

Quantification and statistical analysis: All statistical details, including the study type, population size, and underlying distribution assumptions, for each study are provided in the figure legends and throughout the main text. For statistical analysis, MATLAB was used. Significance is defined at a P-value of 0.05. No collected data was excluded. Studies did not begin until the function of the device was verified.

### Experimental Results and Additional Examples

A study was conducted to develop and evaluate the exemplary method and system.

Mechanical studies were then conducted to empirically determine the soft device's bending stiffness and reliability. First, the device was bent through 180° over a 3 cm radius via a Mark-10 ESM303 Advanced Motorized Test Stand. An image of the device seamlessly bending over this radius is shown in FIG. 7A. The stiffness was calculated as a function of the board's tip displacement and the resultant stiffness of <0.1 N/m indicates exceptional flexibility (FIG. 7B). Second, the device was assessed in bending to 160° over the same radius for 100 cycles. The reduced angle was chosen due to limitations in the testing setup, not an assumption about the device's reliability. Images of the test setup are shown in FIGS. 7C-7E. During the 100 bending cycles, PPG signals were wirelessly transmitted via Bluetooth to an Android receiver. As shown in FIG. 7F, the signals maintained excellent quality throughout the recording. Sample signals at the beginning (FIG. 7G) and end (FIG. 7H) of the trial indicate no difference in electrical functionality.

Next, finite element analysis (FEA) was conducted to simulate the board's compression into the skin to verify that the proposed elastomer dampening system yields a fully conformal contact. When a linear displacement is applied, the simulated board seamlessly conforms to the skin when a skin stress of only 500 kPa is achieved, which is far below the range necessary for vessel occlusion or pain and easily achieved with force applied by a tensed tape [27]. Results of this FEA experiment are shown in FIGS. 7I-7K and compared to a simulation of a traditional polyimide flex PCB that cannot conform to the skin at the same applied stress.

Finally, the device was designed specifically to reduce motion artifacts in sternal PPG, which is the main limiting factor preventing traditional rigid systems from utilizing this anatomical region. Here, it was assumed that non-conformal motion of the board will primarily be caused when the skin bends, stretches, translates laterally, undergoes torsion, or experiences surface waves. However, stretching is likely to dominate, especially during respiration. In this model, the highly flexible board is well suited to handle concave bending and stretching and maintain conformality because of the unique elastomeric dampening design. When the skin stretches, the tension in the tape increases, causing the pressure applied to increase, further pushing the board into conformal contact with the skin. When the user's skin folds inwards, like when a user hugs, for instance, the highly flexible circuit can seamlessly bend with the skin, and the dampening in the elastomer prevents the applied PPG pressure from becoming excessive. Because the tape is uniaxially stretchable, it easily allows for breathing motion in the horizontal plane while limiting torsion, although the circuit's flexibility could likewise handle skin torsion. Before conducting trials in human subjects, the device was tested for biocompatibility by being worn for 3 consecutive days and observing whether any skin irritation occurred. The results are shown in FIGS. 7L-7M, which shows that only very minor skin reddening was noted, even after 3 days of continuous wear. This indicates a high degree of biocompatibility.

Human trials. The first human-subjects trial was conducted to assess the hypothesis that the soft mechanics innovations introduced here can improve signal quality and reduce motion artifacts compared to a rigid device. Signals were simultaneously recorded in both a soft device and a rigid alternative with identical components and footprint during skin perturbations. The magnitude and duration of motion artifacts is increased in the rigid device compared to the soft device. All human subject details are provided in TABLE 1, TABLE 2, and TABLE 3.

TABLE 1 shows a summary of human subjects who volunteered for the location optimization study.

TABLE 1

| Subject | Age | BMI | Gender | Race |
|---|---|---|---|---|
| 1 | 24 | 19 | M | White |
| 2 | 50 | 29 | F | White |
| 3 | 28 | 28 | M | African American |
| 8 | 28 | 24 | M | Middle-Eastern |
| 5 | 51 | 40 | M | White |

TABLE 2 shows a summary of human subjects who volunteered for the vasoconstriction validation study.

TABLE 2

| Subject | Age | BMI | Gender | Race |
|---|---|---|---|---|
| 1 | 24 | 19 | M | White |
| 2 | 32 | 24 | M | White |
| 3 | 50 | 29 | F | White |
| 4 | 28 | 28 | M | African American |
| 5 | 35 | 24 | M | Asian |
| 6 | 22 | 26 | M | White |
| 7 | 20 | 25 | M | Middle-Eastern |
| 8 | 28 | 24 | M | Middle-Eastern |
| 9 | 51 | 40 | M | White |

TABLE 3 shows a summary of human subjects who volunteered for the overnight apnea comparison study.

TABLE 3

| Subject | Age | BMI | Gender | Race | Stop-Band/PSQI/ESS/ISI | OSA |
|---|---|---|---|---|---|---|
| 1 | 31 | 23.2 | M | White | Low/9/0/9 | Mild (5 < x < 15) |
| 2 | 35 | 29.3 | M | White | Low/7/9/7 | Moderate (15 < x < 30) |
| 3 | 59 | 35.2 | M | White | High/7/4/3 | Moderate-Severe |
| | | | | | | (25 < x < 40) |
| 4 | 35 | 27.6 | M | Asian | Low/6/3/7 | Mild (5 < x < 15) |
| 5 | 51 | 39.4 | M | White | Medium/6/4/7 | Moderate (15 < x < 30) |

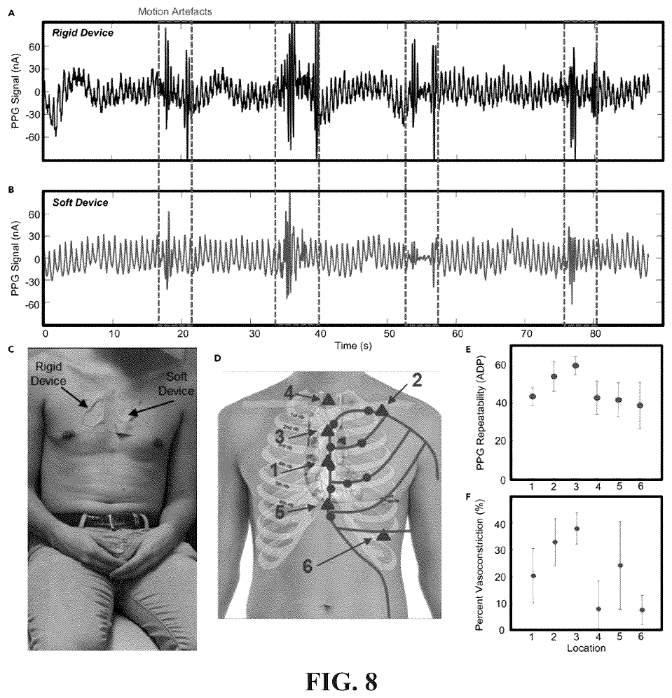

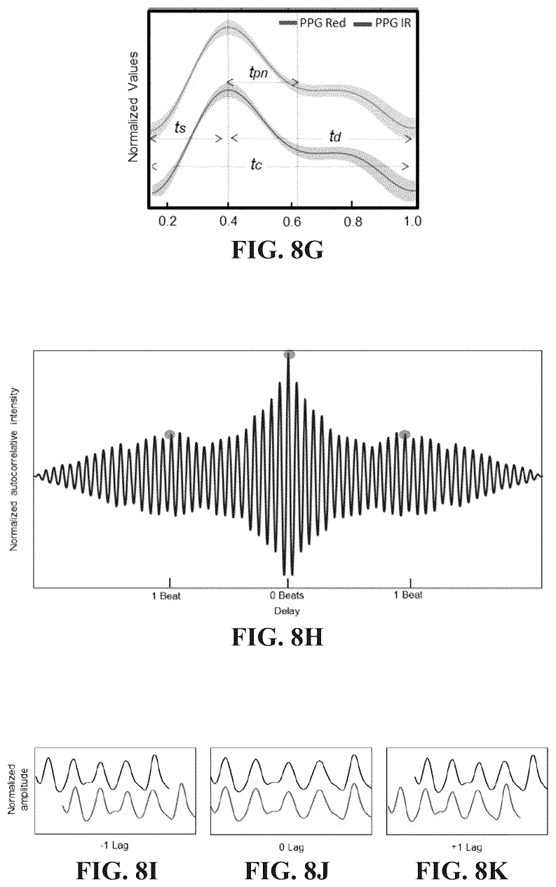

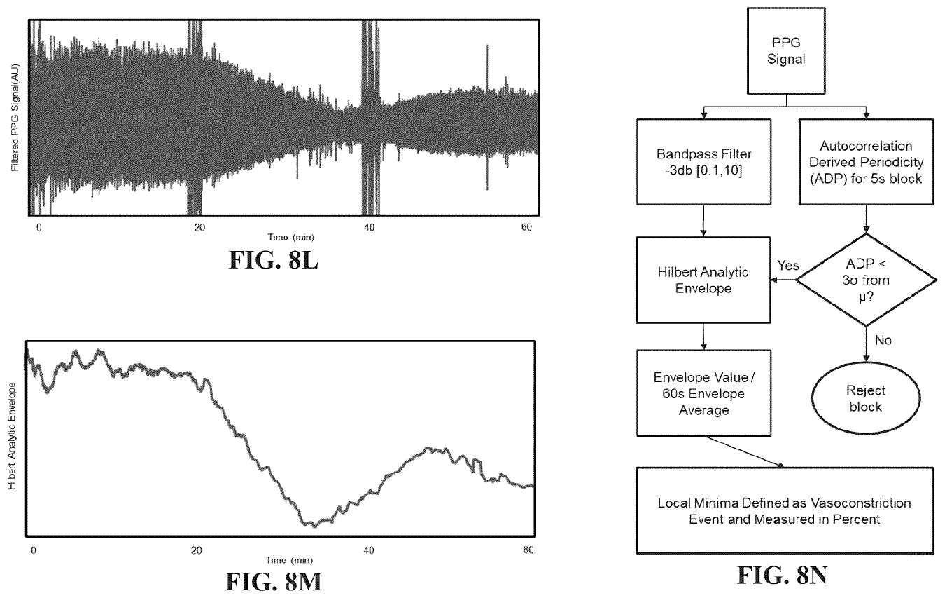

The results are shown in FIGS. 8A-8B, which show comparison unfiltered PPG data between the rigid and soft devices, respectively. The experimental setup is also shown in FIG. 8C. After the optimal device mechanics were determined, the sensing performance was validated with human subjects, and the optimal anatomical region on the chest was identified. Multiple subjects performed three rounds of controlled breath holds of 30 s in duration while wearing the soft device on six different sternal regions, as shown in FIG. 8D. In a real-life clinical application, it is unlikely that the device placement could be precisely guaranteed; thus, the soft device was placed solely based on visual inspection with the aid of the illustration in FIG. 8D. In this illustration, the major vessels are shown in red, and areas of high angiosome density are shown in green. For each trial, the PPG signals were assessed for repeatability and percent vasoconstriction (FIG. 8G). The PPG repeatability is defined by the ratio of the signal autocorrelation at zero delay to the average of the autocorrelation at delays of one, two, and three beats. This metric captures how similar a delayed copy of the signal is to the original, thus providing a surrogate measure for the signal-to-noise ratio for complicated signals. The process is summarized in FIGS. 8H-8K.

The percent vasoconstriction is defined as the local minima divided by the 60 s running average of the signal's Hilbert analytic envelope. The envelope calculation is summarized in FIGS. 8L-8M, and it captures the magnitude of the signal over time. Here, this Hilbert analytic envelope magnitude is used to determine the subcutaneous arterial perfusion in the skin, a contraction of which is considered a vasoconstriction.

The overall processing flow is summarized in FIG. 8N. Note that the periodicity is used to reject high noise beats from assessment in the determination of vasoconstriction. Across all sites, an acceptable PPG repeatability was demonstrated as the subject rested in a supine position. A sample ensemble of PPG waves during a 2-minute recording from location 3 is shown in FIG. 8G, clearly indicating a high ability to detect crucial PPG waveform parameters and timing intervals.

The overall repeatability results are shown in FIG. 8E, demonstrating that location 3 had the highest repeatability. AZ-test between locations 2 and 3 shows that the two distributions are statistically different, with a Z-score of 4.21. The average vasoconstriction magnitude, reported in percent, is captured in FIG. 8F.

Here, the vasoconstriction distributions for locations 2 and 3 show the marginal statistical difference, with a z Score of 2.41 corresponding to a p-value of 0.082, for the hypothesis that both datasets are derived from the same statistical distribution. Based on the increased repeatability and marginally increased vasoconstriction, location 3 was selected as the optimal device placement, although location 2 was also deemed acceptable. However, location 2 is more susceptible to motion artifacts due to the curved nature of the collarbone, and the device would be visible under most garments. Therefore, location 3 is deemed the most appropriate measurement location for continuous vasoconstriction detection.

Vasoconstrictionsfrom controlled studies: After the device placement was optimized, additional controlled experiments in human subjects were con-ducted to validate the system's efficacy in endogenous and exogenous vasoconstriction detection. A breath-hold may induce vasoconstriction when chemoreceptors identify a decrease in oxygen saturation, but this response can vary greatly depending on a subject's unique physiology. FIG. 9A shows the device placed on a subject's sternum during one simple breath hold test where subjects held their breath for 30 s, and FIGS. 9B-9C show representative vasoconstrictions from two different subjects. In these tests, both the red and infrared (IR) PPG waveforms are collected. Because the penetration distance of light into the skin increases with wavelength based on the Beer-Lambert Law, this allows us to report both the dermis (red) and hypodermis (IR) perfusion. As shown in the figures, the vasoconstrictions manifest differently in each subject. In FIG. 9B, the constriction is characterized by a rapid onset and a rapid termi-nation. In FIG. 9C, the constriction is rapid, but the recovery is elongated. The constrictions also manifest similarly in both the red and IR signals. However, the IR signal has notably higher amplitude and signal repeatability (increased repeatability by 41% SNR on average across 9 subjects). Thus, the IR signal was used for all further analysis. Regarding the differences in vasoconstriction morphologies, it was hypothesized that each subject would react differently to the breath hold based on their individual physiologies. In both cases, the soft patch can accurately capture the subject's vasoconstriction. Motion artifacts occurred during the termination of the breath hold, which is to be expected due to the aggressive chest motion. Still, the device immediately regained excellent conformality and recorded unperturbed PPG waveforms.

The vasoconstriction results for all 9 subjects across 5 trials are summarized in FIG. 9D, which shows that the device is capable of detecting vasoconstrictions across a wide range of vasoconstriction percentages and subject variabilities. Interestingly, varying percent blood flow constrictions were observed both be-tween subjects and for the same subject across different trials. Given that there is no clinical comparison here, an additional study was conducted to validate our ability to differentiate between small, medium, and large constrictions. It may be concluded here, however, that the device is capable of a binary distinction between events and no events, as vasoconstrictions were observed in all 5 trials for 5 subjects and 91% of trials overall (41/45) in all 9 subjects. Next, the sensor's ability to detect exogenous vasoconstrictions was validated by considering the case of changes in core body temperature. A subject was instructed to remain outdoors at a 28° C. ambient temperature for 20 min before entering a room at 15° C. The subject then returned outdoors, allowing for an assessment of constriction and dilation due to ambient temperature changes. The results of this experiment are shown in FIG. 9E, where the constriction and dilation are clearly shown. Thus, this study demonstrates a high ability to detect endogenous and exogenous vasoconstrictions in healthy control subjects.

Assessment of vasoconstrictions in subjects with sleep apnea: Finally, the soft device's ability to detect naturally occurring vasoconstrictions in patients with sleep apnea resulting from sympathetic arousals during sleep was studied. Eight nights of sleep were recorded across six subjects. The subject who exhibited the highest physiological propensity for vasoconstriction, as deter-mined by their average vasoconstriction magnitude on the first night, was selected to perform a second night with a clinical grade comparison device, the WatchPAT probe. FIG. 10A shows a representative hour of sleep showing the WatchPAT signal compared to the soft device's PPG signal. The filtered waveforms correspond to the upper three signals and the Hilbert envelope the lower, respectively. Here, each of the apnea events is automatically classified based on WatchPAT's proprietary algorithm, which also considers heart rate, respiration rate, and actigraphy. In FIG. 10A, the times corresponding to each identified event are highlighted in red. As shown in the figure, there is a high agreement between vasoconstrictions in the WatchPAT signal and both the red and IR PPG signals from the soft device. However, the agreement is not perfect, both with respect to the constriction magnitude and the presence of constriction. Given that there is no clinical comparison device that measures vasoconstriction from the chest, this result is potentially due to variances in physiological response based on anatomical location. Despite any such differ-ences, the soft device and WatchPAT probe do demonstrate a clear correlation.

This correlation is quantified in FIG. 10B, which shows a comparison of vasoconstrictions in both the soft device and WatchPAT comparison when discretized into small (0%-15%), medium (15%-30%), and large (30%-50%) bins. Here, the soft device demonstrates a 78% agreement with the WatchPAT comparison in identifying systemic vasoconstrictions, according to the above schema. The specific nature of each missed event provides further support for the soft device's detection efficacy. As FIG. 10B demonstrates all the missed events occurred because WatchPAT detected a larger vasoconstriction than the soft device. The statistical relationship between the two datasets is further investigated in the Bland-Altman plot in FIG. 10C and the correlation plot in FIG. 10D, respectively. In FIG. 10C, the mean is shown to skew toward a higher observed vasoconstriction in the WatchPAT device than the soft device, but the variance remains consistent as the mean increases. The limits of agreement are summarized in the figure. Next, the correlation plot in FIG. 10D shows a highly linear relationship between the two devices' simultaneous vasoconstriction re-cordings across all 36 events, resulting in an r² of 0.74. Furthermore, FIG. 10E shows the distribution of all events for both the soft device and the red and IR channels of the soft device (the blue line indicates average), and it demonstrates that the WatchPAT device, whether due to physiological differences or variations in applied pressure, has a higher average estimate. Therefore, it is highly likely that the remaining 22% inaccuracy is due to this systemic bias rather than a lack of detection efficacy. This result compares very favorably with alternative wearable vasoconstriction monitoring approaches. In addition to this study, data were collected in seven additional nights, and the overall vasoconstriction response is summarized in FIG. 10F, and the individual responses are shown in FIG. 10G. Although each patient had a varying degree of vasoconstrictive response, the soft device is capable of detecting constrictions in all subjects. An image of the soft device placement on a subject is shown in FIG. 10H.

All materials used in this study are shown in TABLE 4.

TABLE 4

| | | |
|---|---|---|
| Reagent or | | |
| Resource | Source | Identifier |

Chemicals, peptides, and recombinant proteins

| | | |
|---|---|---|
| Eco-Flex 00-30 | Smooth-On | 00-30 |
| PDMS elastomer | Sylgard | 184 |
| PDMS curing agent | Sylgard | N/A |
| Easy Release 2831 | Mann | 2831 |
| Acetone 99.5+% | Alfa Aesar | 99.5+% |
| Isopropyl Alcohol | Lab Chem | Isopropyl Alcohol |
| PI2610 | HD microsystems | 2610 |
| Copper | Sigma Aldrich | 326445 |
| Solder Paste | Digikey | SMDLTLFP10T5-ND |

### DISCUSSION

Vasoconstriction and vasodilation refer to the narrowing and widening of arterial pathways. This process is controlled by the concentration of calcium ions present in arterial smooth muscle cells. The body uses it as a crucial mechanism to regulate blood pressure, reduce hemorrhaging, maintain core body temperature and partition blood flows [1], [2]. In addition to being a key player in how a healthy body maintains homeostasis, vasoconstriction is also a marker of several health conditions, like sickle cell disease, shock, migraines, asthma, glaucoma, rosacea, and chronic stress and exacerbator of several others, like Raynaud's phenomenon, hypertension, headaches, and coronary artery disease [3]-[7]. Furthermore, induced vasodilation is one of the most effective and well-studied means of medically reducing blood pressure, forming the basis of numerous pharmaceutical treatments. Because vasoconstriction is central to so many physiological processes, it is crucial to measure and challenging to isolate. One mechanism for vasoconstriction detection is to measure PPG signals, where an optical sensor is used to determine the pulsatile blood flow in an artery [9].

However, PPG is rarely employed to measure vasoconstriction today because the anatomical regions where PPG is typically measured, like the fingers, ear, forehead, and foot, are perfused primarily by capillary structures, where venous pooling and respiratory modulation produce confounding signals [10], [11]. In addition, these regions are greatly affected by changes in peripheral body temperature [11]. Instead, vasoconstriction is primarily measured with obtrusive systems, like laser Doppler flowmetry or high-pressure cuffs that eliminate venous pooling [12], [13]. Thus, a tremendous opportunity to characterize patient health and detect disease is foregone. If one could measure vasoconstriction in real-time, it would be possible to assess the effectiveness and adjust the dosage of blood pressure medication, detect serious health conditions, like a sickle cell attack, before it occurs, identify long-term trends in sympathetic neural activity and patient stress, and better understand an individual's unique response to hormonal medication [14].

Although traditional PPG monitoring sites are incompatible with continuous vasoconstriction detection, the chest is an attractive alternative because the subcutaneous microcirculation is driven in principle by central angiosomes, which exhibit a much lesser venous pooling and respiratory effect than capillary vascularization [15]-[17]. However, no commercial device has successfully measured vasoconstriction from the chest because the area is poorly perfused, resulting in a lower signal amplitude. The chest surface morphology is highly subject-dependent and nonlinear. Thus, it is challenging to apply sufficient pressure, unlike in a finger probe, and quality skin-device contact is difficult to maintain because of motion artifacts. [10]-[15]. Several attempts have been made to measure PPG from the chest, but the rigid sensing materials employed have proven incapable of maintaining sufficient skin-device contact [18], [19]. Likewise, no soft sensing system has successfully implemented the necessary pressure on the PPG unit to induce high enough optical coupling between the sensor and the skin [20]. There are a number of rigid and bulky devices that monitor vasoconstriction [12], [13], [21]-[26].

In contrast, the exemplary system employs a wireless, soft sternal patch with optimized skin-like electronics finely tuned to measure vasoconstriction from the sternum continuously. The study performed analytical, computational, and empirical evaluations of soft materials and mechanics to provide a pressure application and elastomer-dampening system capable of mitigating motion artifacts in the PPG signal from the chest and producing a highly conformal skin-device contact.

First, a human subject study determined that the mid-sternum is the optical chest location for vasoconstriction detection, and the final device optimization was tuned for this region. Next, vasoconstriction detection was validated during controlled breath holds and core body temperature modulation in healthy subjects, demonstrating high detection efficacy across all subjects. Finally, in overnight trials, vasoconstriction induced by sympathetic arousals in patients with sleep apnea was detected, and the device demonstrated high agreement with the FDA-approved, commercial WatchPAT system. The fundamental studies in chest-based PPG monitoring are valuable for their immediate medical value and general applicability to the study of soft sensors and mechanics. Furthermore, the results reported here are of general interest for PPG recording in a wide range of traditionally unfavorable anatomical regions.

### Example Trained Machine Learning Model

Machine Learning. In some embodiments, a machine learning model can be employed to evaluate the vasoconstriction response, e.g., for a given clinical application, the analysis system can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Example Computing System

It should be appreciated that the logical operations described above and in the appendix can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### CONCLUSION

Each and every feature described herein, and each and every combination of two or more of such features, is included within the scope of the present invention, provided that the features included in such a combination are not mutually inconsistent.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The following patents, applications, and publications, as listed below and throughout this document, are hereby incorporated by reference in their entirety herein.

### REFERENCES

[1] Paal, P., and Strapazzon, G. (2018). Thermoregulation: from basic neuroscience to clinical neurology, Part II. Handb. Clin. Neurol.
[2] Wikel, S. (2017). Arthropod modulation of wound healing. Arthropod Vector: Controller of Disease Transmission 2, 31-50. https://doi.org/10.1016/B978-0-12-805360-7.00003-4.
[3] Khaleel, M., Puliyel, M., Shah, P., Sunwoo, J., Kato, R. M., Chalacheva, P., Thuptimdang, W., Ketterich, J., Wood, J. C., Tsao, J., et al. (2017). Individuals with sickle cell diseasehave a significantly greater vasoconstriction response to thermal pain than controls and have significant vasoconstriction in response to anticipation of pain. Am. J. Hematol. 92, 1137-1145. https://doi.org/10.1002/ajh. 24858.
[4] Tanoue, A., Nasa, Y., Koshimizu, T., Shinoura, H., Oshikawa, S., Kawai, T., Sunada, S., Takeo, S., and Tsujimoto, G. (2002). The a.1Dadrenergic receptor directly regulates arterial blood pressure via vasoconstriction. J. Clin. Invest. 109, 765-775. https://doi.org/10.1172/jci200214001.
[5] Phan, T. X., Ton, H. T., Gulyas, H., P6rszasz, R., T6th, A., Russo, R., Kay, M. W., Sahibzada, N., and Ahern, G. P. (2020). TRPV1 expressed throughout the arterial circulation regulates vasoconstriction and blood pressure. J. Physiol. 598, 5639-5659. https://doi.org/10. 1113/JP279909.
[6] Abdulle, A. E., van Roon, A. M., Smit, A. J., Pasch, A., van Meurs, M., Bootsma, H., Bakker, S. J. L., Said, M. Y., Fernandez, B. O., Feelisch, M., et al. (2019). Rapid free thiol rebound is a physiological response following cold-induced vasoconstriction in healthy humans, primary Raynaud and systemic sclerosis. Physiol. Rep. 7, e14017. https://doi.org/10.14814/phy2.14017.
[7]. Hearon, C. M., Jr., and Dinenno, F. A. (2019). Escape, lysis, and feedback: endothelial modulation of sympathetic vasoconstriction. Curr. Opin. Pharmacol. 45, 81-86. https://doi. org/10.1016/j.coph.2019.04.002.
[8] Bellew, S. D., Johnson, K. L., Nichols, M. D., and Kummer, T. (2018). Effect of intranasal vasoconstrictors on blood pressure: a randomized, double-blind, placebocontrolled trial. J. Emerg. Med. 55, 455-464. https://doi.org/10.1016/j.jemermed.2018. 07.004.
[9] Guess, M., Zavanelli, N., and Yeo, W. H. (2022). Recent advances in materials and flexible sensors for arrhythmia detection. Materials 15, 724. https://doi.org/10.3390/ma 15030724.
[10] Tur, E., Tur, M., Maibach, H. I., and Guy, R. H. (1983). Basal perfusion of the cutaneous microcirculation: measurements as a function of anatomic position. J. Invest. Dermatol. 81, 442-446. https://doi.org/10.1111/1523-1747. ep12522619.
[11] Nilsson, L., Goscinski, T., Kalman, S., Lindberg, L. G., and Johansson, A. (2007). Combined photoplethysmographic monitoring of respiration rate and pulse: a comparison between different measurement sites in spontaneously breathing subjects. Acta Anaesthesiol. Scand. 51, 1250-1257. https://doi.org/10.1111/j.1399-6576.2007. 01375.x.
[12] Wang, L., Ansari, S., Cai, Y., McCracken, B., Hakam Tiba, M., Ward, K. R., Najarian, K., and Oldham, K. R. (2022). Tracking peripheral artery motion and vascular resistance with a multimodal wearable sensor under pressure perturbations. J. Biomech. Eng. 144, 081003. https://doi.org/10.1115/1.4053399.
[13] Schwartz, K. S., Theis, E. N., Bunting, K., McCaughey, R. A., and Lang, J. A. (2022). Laser speckle contrast imaging and laser Doppler flowmetry reproducibly assess reflex cutaneous vasoconstriction. Microvasc. Res. 142, 104363. https://doi.org/10.1016/j.mvr. 2022.104363.
[14] Chalacheva, P., Khaleel, M., Sunwoo, J., Shah, P., Detterich, J. A., Kato, R. M., Thuptimdang, W., Meisel man, H. J., Sposto, R., Tsao, J., et al. (2017). Biophysical markers of the peripheral vasoconstriction response to pain in sickle cell disease. PLoS One 12, e0178353. https://doi.org/10.1371/journal.pone.0178353.
[15] Nilsson, L. M. (2013). Respiration signals from photoplethysmography. Anesth. Analg. 117, 859-865. https://doi.org/10.1213/ANE.0b013e31828098b2.
[16] Mannheimer, P. D., O'Neil, M. P., and Konecny, E. (2003). The influence of larger subcutaneous blood vessels on pulse oximetry. J. Clin. Monit. Comput. 18, 179-188. https://doi.org/10.1023/B:JOCM. 0000042924.16052.5e.
[17] Longmore, S. K., Lui, G. Y., Naik, G., Breen, P. P., Jalaludin, B., and Gargiulo, G. D. (2019). A comparison of reflective photoplethysmography for detection of heart rate, blood oxygen saturation, and respiration rate at various anatomical locations. Sensors 19, 1874. https://doi.org/10.3390/s19081874.
[18] Chreiteh, S. (2016). Investigation of Sternal Photoplethysmography—Design of a Vital Sign Patch (Technical University of Denmark).
[19] Chreiteh, S. S., Saadi, D. B., Belhage, B., Nabipour, N., Hoppe, K., and Thomsen, E. V. (2016). A clinical study of short-term sternal photoplethysmography: recordings from patients with obstructive airways diseases. In Proceedings of the Annual International Conference of the IEEE Engineering in Medicine and Biology Society (EMBS. Institute of Electrical and Electronics Engineers Inc).
[20] Naslund, E., Lindberg, L. G., Lund, I., NaslundKoch, L., Larsson, A., and Frithiof, R. (2020). Measuring arterial oxygen saturation from an intraosseous photoplethysmographic signal derived from the sternum. J. Clin. Monit. Comput. 34, 55-62. https://doi.org/10.1007/s10877-019-00289-w.
[21] Lai, M., van der Stel, S. D., Groen, H. C., van Gastel, M., Kuhlmann, K. F. D., Ruers, T. J. M., and Hendriks, B. H. W. (2022). Imaging PPG for in vivo human tissue perfusion assessment during surgery. J. Imaging 8, 94. https://doi.org/10.3390/jimaging8040094.
[22] Venema, B. (2021). Reflective arterial pulse oximetry for new measuring sites and longterm assessment of dermal perfusion. Studies in Skin Perfusion Dynamics, 79-91.
[23] Chan, M., Ganti, V. G., Heller, J. A., Abdallah, C. A., Etemadi, M., and lnan, O. T. (2021). Enabling continuous wearable reflectance pulse oximetry at the sternum. Biosensors 11, 521. https://doi.org/10.3390/bios11120521.
[24] Cote, G. L., Adhikari, P., Magana, I. B., and O'Neal, P. D. (2014). Multi-wavelength pulse plethysmography for real-time drug delivery monitoring. In Optical Diagnostics and Sensing XIV: Toward Point-of-Care Diagnostics. https://doi.org/10.1117/12. 2040064.
[25] Theis, E., Schwartz, K., Bunting, K., Mccaughey, R., and Lang, J. (2021). Reproducibility and effect of sampling depth on laser Doppler flowmetry measures during the reflex cutaneous vasoconstriction response. FASEB j. 35.
[26] Korkuyu, E., Duzlu, M., Karamert, R., Tutar, H., Yilmaz, M., <:;ift˜i. B., and Guven, S. F. (2015). The efficacy of Watch PAT in obstructive sleep apnea syndrome diagnosis. Eur. Arch. Oto-Rhino-Laryngol. 272, 111-116. https://doi.org/10.1007/s00405-014-3097-0.
[27] Zavanelli, N., Kim, H., Kim, J., Herbert, R., Mahmood, M., Kim, Y. S., Kwon, S., Bolus, N. B., Torstrick, F. B., Lee, C. S. D., and Yeo, W. H. (2021). At-home wireless monitoring of acute hemodynamic disturbances to detect sleep apnea and sleep stages via a soft sternal patch. Sci. Adv. 7, eab14146. 28. Zavanelli, N., Kim, J., and Yeo, W. H. (2021). Recent advances in high-throughput nanomaterial manufacturing for hybrid flexible bioelectronics. Materials 14, 2973. https://doi.org/10.3390/ma14112973.
[29] Zavanelli, N., and Yeo, W. H. (2021). Advances in screen printing of conductive nanomaterials for stretchable electronics. ACS Omega 6, 9344-9351. https://doi.org/10.1021/acsomega.1 c00638.

What is claimed:

1. A system comprising:
a processor; and
a memory having instructions stored thereon, wherein the instructions when executed by the processor causes the processor to:
receive a photoplethysmogram (PPG) signal data set acquired by a soft patch comprising a vasoconstriction sensor placed on a chest of a subject; and
determine if the subject is experiencing vasoconstriction using the the PPG signal data set;
wherein the soft patch compresses the vasoconstriction sensor into the chest of the subject.

2. The system of claim 1, wherein the instructions, when executed by the processor, further cause the processor to output if the subject is experiencing vasoconstriction in a graphical user interface or a report to be used for an evaluation of cardiovascular health in the subject.

3. The system of claim 1, wherein the soft patch further comprises an electrocardiography sensor and/or a seismocardiography sensor.

4. The system of claim 1, wherein the soft patch is placed on a sternum of the subject.

5. The system of claim 1, wherein the soft patch further comprises an elastomer disposed over the vasoconstriction sensor; and
wherein the elastomer dampens motion artifacts in the PPG signal data caused by movement of the chest of the subject.

6. The system of claim 1, wherein the soft patch includes wireless communication circuitry.

7. The system of claim 1, wherein the instructions, when executed by the processor, further cause the processor to determine via a trained machine learning model if the subject is experiencing sleep apnea.

8. The system of claim 7, wherein the instructions, when executed by the processor, further cause the processor to output if the subject is experiencing sleep apnea in a graphical user interface or a report to be used for an evaluation of sleep apnea in the subject.

9. The system of claim 1, wherein the system is configured as a smartwatch or smartphone.

10. The system of claim 1, wherein the system is implemented in cloud infrastructure.

11. A method comprising:
receiving a PPG signal dataset acquired by a soft patch comprising a vasoconstriction sensor placed on a chest of a subject; and
determining if the subject is experiencing vasoconstriction using the the PPG signal data set;
wherein the soft patch compresses the vasoconstriction sensor into the chest of the subject.

12. The method of claim 11, further comprising outputting if the subject is experiencing vasoconstriction in a graphical user interface or a report to be used for an evaluation of cardiovascular health in the subject.

13. The method of claim 11, wherein the soft patch further comprises an electrocardiography sensor and/or a seismocardiography sensor.

14. The method of claim 11, wherein the soft patch is placed on a sternum of the subject.

15. The method of claim 11, wherein the soft patch further comprises an elastomer disposed over the vasoconstriction sensor; and
wherein the elastomer dampens motion artifacts in the PPG signal data caused by movement of the chest of the subject.

16. The method of claim 11, wherein the soft patch includes wireless communication circuitry.

17. The method of claim 11, further comprising determining via a trained machine learning model if the subject is experiencing sleep apnea.

18. The method of claim 17, further comprising outputting if the subject is experiencing sleep apnea in a graphical user interface or a report to be used for an evaluation of sleep apnea in the subject.

19. A non-transitory computer readable medium having instructions stored thereon, wherein the instructions when executed by a processor causes the processor to:
receive a photoplethysmogram (PPG) signal data set acquired by a soft patch comprising a vasoconstriction sensor placed on a chest of a subject; and
determine if the subject is experiencing vasoconstriction using the the PPG signal data set;
wherein the soft patch compresses the vasoconstriction sensor into the chest of the subject.

20. The computer readable medium of claim 19, wherein the instructions, when executed by the processor, further cause the processor to output if the subject is experiencing vasoconstriction in a graphical user interface or a report to be used for an evaluation of cardiovascular health in the subject.
